(19) United States
(12) Patent Application Publication
(10) Pub. No.: US 2006/0167535 A1
(43) Pub. Date: Jul. 27, 2006

(54) Deflectable coronary sinus lead delivery catheter

(76) Inventor: Eric T. Johnson, Temecula, CA (US)

Correspondence Address: JASON R. KRAUS;FAEGRE & BENSON, LLP, 2200 WELLS FARGO CENTER, 90 SOUTH SEVENTH STREET, MINNEAPOLIS, MN, 55402-3901, US

(21) Appl. No.: 11/041,359

(22) Filed: Jan. 24, 2005

(51) Int. Cl. A61N 1/05; A61N 1/362; A61M 31/00

(52) U.S. Cl. 607/122; 604/95.04; 607/9

(57) ABSTRACT

A catheter assembly for cannulating the coronary sinus of the heart includes a flexible, elongate shaft having a proximal end extending to a distal end. A central lumen and a secondary lumen extend through the shaft. The distal end is preformed with a first curved segment and a second curved segment extending distally from the first curved segment. A deflecting mechanism is positioned in the secondary lumen and is operable to deflect the first curved segment from a first configuration to a second configuration while allowing the second curved segment to remain in a first configuration. The deflecting mechanism may be a tensioning member fixed to the shaft and extending through the secondary lumen or a rigid member slidably received in the secondary lumen.

### CROSS REFERENCES

Reference is hereby made to the following commonly assigned U.S. Patent Applications, which are incorporated herein by reference: application Ser. No. 10/916,353 filed Aug. 11, 2004 and entitled “Coronary Sinus Lead Delivery Catheter” and application Ser. No. 10/916,148 filed Aug. 11, 2004 and entitled “Right-Side Coronary Sinus Lead Delivery Catheter.”

### TECHNICAL FIELD

The present invention is related to implantable medical devices, and in particular to guide catheter assemblies for delivery of cardiac pacing leads.

### BACKGROUND

Guiding catheters are instruments that allow a physician to access and cannulate vessels in a patient's heart for conducting various medical procedures, including venography and implantation of cardiac leads. Cannulating heart vessels requires navigating a small-diameter flexible guide catheter through the vasculature into the heart, and then into a destination heart vessel. Once the destination heart vessel is reached, the catheter acts as a conduit for insertion of payloads, for example, pacing leads, into the vessel.

One commonly accessed destination vessel for placement of cardiac pacing leads is the coronary sinus. While access to the coronary sinus is typically gained through the left subclavian vein, the coronary sinus may also be accessed through the right subclavian vein. Guiding catheter systems are often configured with a pre-shaped profile that is optimized for the intended vessel destination.

There is a need for a lead delivery system and method having improved maneuverability to allow accurate and rapid lead implantation and anchoring in selected vessels, such as the coronary sinus or sub-branches of the coronary sinus.

### SUMMARY

According to one embodiment, the present invention is a catheter assembly for cannulating a coronary sinus of the heart. The catheter assembly includes a flexible, elongated shaft having a proximal end, a distal end, a central lumen and a secondary lumen that is co-axial with the central lumen. The distal end is preformed with a first curved segment and a second curved segment extending distal to the first curved segment. The catheter assembly further includes a tensioning member positioned in the secondary lumen and coupled to the shaft proximal to the second curved segment for deflecting the first curved segment from a first configuration to a second configuration while the second curved segment remains in a first configuration.

According to another embodiment the present invention is a system for performing cardiac rhythm management on a heart. The system includes a pulse generator, a lead and a catheter assembly. The lead has a proximal end coupled to the pulse generator, a distal end coupled to the heart and an electrode electrically coupled to the pulse generator at the distal end of the lead. The catheter assembly includes a flexible, elongated shaft having a proximal end, a distal end, a central lumen, and a secondary lumen co-axial with the central lumen. The distal end is preformed with a first curved segment and a second curved segment extending distal to the first curved segment. The catheter assembly further includes a deflecting means for deflecting the first curved segment from a first configuration to a second configuration.

According to yet another embodiment, the present invention is a method of cannulating the coronary sinus. A catheter assembly is provided of the type including a shaft having a proximal end, a distal end having a first curved segment and a second curved segment extending distally therefrom, a central lumen for receiving a pacing lead and a deflecting mechanism positioned in a secondary lumen of the shaft. The distal end of the shaft is inserted into an access vessel to the heart. The catheter assembly is advanced distally along the access vessel. The first curved segment is deflected to reposition the shaft while allowing the second curved segment to remain in substantially the same configuration. The coronary sinus is accessed with the shaft. A pacing lead is advanced into the coronary sinus through the central lumen.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a partial sectional view of a portion of the vasculature and a catheter assembly in accordance with one embodiment of the present invention.

FIG. 2A is a side sectional view of a distal portion of the catheter assembly of FIG. 1.

FIG. 2B is a cross-sectional view of the catheter assembly of FIG. 2A taken along line B-B.

FIG. 3 is a simplified side view of the catheter assembly of FIG. 2A in various deflected and undeflected positions according to one embodiment of the present invention.

FIG. 4 is a partial sectional view of the heart and a portion of the catheter assembly of FIG. 3 in deflected and undeflected positions.

FIG. 5 is a flowchart illustrating a method of cannulating the coronary sinus of the heart according to one embodiment of the present invention.

FIG. 6A is a side sectional view of a distal portion of a catheter assembly in accordance with another embodiment of the present invention.

FIG. 6B is a cross-sectional view of the catheter assembly of FIG. 6A taken along line B-B.

FIG. 7 is a simplified side view of the catheter assembly of FIG. 6A in various deflected and undeflected positions according to one embodiment of the present invention.

FIG. 8 is a flow chart illustrating a method of cannulating the coronary sinus of the heart according to one embodiment of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

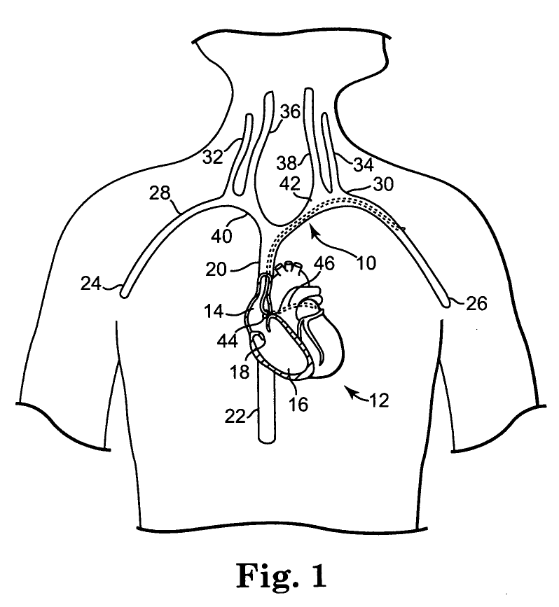

FIG. 1 shows a catheter assembly 10 deployed in a human heart 12 according to one embodiment of the present invention. The heart 12 includes a right atrium 14 and a right ventricle 16 separated by a tricuspid valve 18. During normal operation of the heart 12, deoxygenated blood is fed into the right atrium 14 through the superior vena cava 20 and the inferior vena cava 22. The major veins supplying blood to the superior vena cava 20 include the right and left axillary veins 24 and 26, which flow into the right and left subclavian veins 28 and 30. The right and left external jugular veins 32 and 34, along with the right and left internal jugular veins 36 and 38, join the right and left subclavian veins 28 and 30 to form the right and left brachiocephalic veins 40 and 42. The right and left brachiocephalic veins 40 and 42 combine to flow into the superior vena cava 20.

As shown in FIG. 1, the catheter assembly 10 enters the vascular system through the left subclavian vein 30, extends through the left brachiocephalic vein 42 and the superior vena cava 20, and enters the right atrium 14. As further shown, catheter assembly 10 extends through the coronary sinus ostium 44 so that the catheter assembly 10 is located in the coronary sinus 46. In other embodiments of the present invention, the catheter assembly 10 enters the vascular system through the left and right axillary veins 24 and 26, the left and right external jugular veins 32 and 34, the left and right internal jugular veins 36 and 38, the left and right brachiocephalic veins 40 and 42 or the right subclavian vein 28. The catheter assembly 10 may be a guide catheter serving as a conduit for delivery and positioning of a payload, such as a cardiac lead (not shown) into the heart 12.

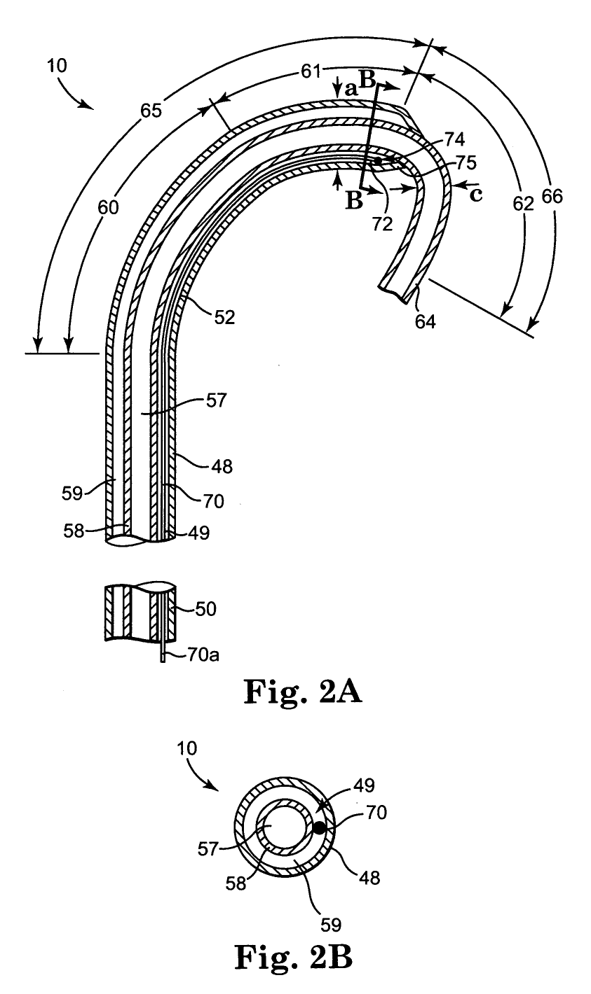

FIGS. 2A and 2B illustrate the catheter assembly 10 of FIG. 1 in accordance with one embodiment of the present invention. The catheter assembly 10 includes a flexible, elongated shaft 48 and a deflecting mechanism 49 for deflecting a portion of the shaft 48. The shaft 48 extends from a proximal end 50 to a distal portion 52. As shown in FIG. 2A, shaft 48 has an open central lumen 57 extending therethrough for receiving a payload, such as a cardiac lead (not shown). An inner shaft 58 is positioned in the central lumen 57, defining an annular space, or secondary lumen 59, that is exterior to and coaxial with the central lumen 57. The proximal end 50 of the shaft 48 is generally straight or unbiased, while the distal portion 52 of the shaft 48 is advantageously pre-shaped with a series of arcs or curves providing an optimized geometry for locating specific vascular features (e.g., the coronary sinus 46). The distal portion 52 includes a first curved segment 60 extending distally from the proximal end 50, a second curved segment 61 extending distally from the first segment 60, and a third curved segment 62 extending distally from the second segment 61. The shaft 48 terminates at a tip 64 extending distal to the third segment 62.

The first segment 60 and second segment 61 characterize a shaft proximal curve 65 having an overall curvature shaped to cause the shaft 48 to find support from the walls of the right atrium 14 during insertion. The third segment 62 characterizes a shaft distal or fixation curve 66 having an overall curvature shaped to facilitate access to the coronary sinus 46 from the right atrium 14 through the coronary sinus ostium 44. According to other embodiments, the shaft 48 may include additional curved or straight segments to form the proximal curve 65 and the fixation curve 66. Examples of curved shapes for the distal end 52 of the shaft 48 are described in above-identified U.S. patent applications entitled “Coronary Sinus Lead Delivery Catheter” and “Right-Side Coronary Sinus Lead Delivery Catheter.”

The deflecting mechanism 49 is operable to selectively and temporarily bend or flex the catheter assembly 10 to facilitate access of chosen cardiac vessels. The deflecting mechanism 49 is further operable to deflect or change the curvature of the proximal curve 65 without substantially deflecting or changing the curvature of the fixation curve 66.

In one embodiment, the deflecting mechanism 49 includes a tensioning member 70 positioned within the secondary lumen 59. Tensioning member 70 may be a tendon wire, a suture or other similar structure. A distal end 72 of the tensioning member 70 is fixed to the shaft 48 at a fixation location 74 proximal to the fixation curve 66. The tensioning member 70 extends proximally through the secondary lumen 59 so that a proximal end 70a is accessible at the proximal end 50 of the shaft 48. Tension exerted on the tensioning member 70 at the proximal end 70a transmits an axial force to the shaft 48 at the fixation location 74, deflecting the shaft 48 proximal to the fixation location 74. The central lumen 57 remains unobstructed for receipt of a payload, such as a cardiac lead. Alternately, the tensioning member 70 is fixed to the inner shaft 58 at the fixation location 74.

The shaft 48, in one embodiment, has an outer diameter a at the proximal curve 65 of about 0.118 inches. The shaft 48 terminates distal to the fixation location 74 at a tapering region 75, and is sealed to the outer surface of the inner shaft 58 such that the secondary lumen 59 is terminated. A portion of the inner shaft 58 having an outer diameter c of about 0.105 inches protrudes distal to the tapering region 75 and is formed with the fixation curve 66. The tapering region 75 is shown positioned between the proximal curve 65 and the distal curve 66 such that the shaft 48 has the outer diameter a at the proximal curve 65 and the inner shaft 58 has the outer diameter c at the distal curve 66. According to other embodiments, the tapering region 75 is positioned elsewhere on the shaft 48 proximal to the distal curve 66. The shaft 48 outer diameter a may be from about 0.112 to about 0.125 inches, and the inner shaft 58 outer diameter c may be from about 0.103 to about 0.112 inches. According to one embodiment, the shaft 48 outer diameter a is about one French larger than the inner shaft 58 outer diameter c. For example, in various embodiments, the shaft 48 outer diameter a is about 9 French and the inner shaft 58 outer diameter c is about 8 French, the shaft 48 outer diameter a is about 8 French and the inner shaft 58 outer diameter c is about 7 French, or the shaft 48 outer diameter a is about 7 French and the inner shaft 58 outer diameter c is about 6 French.

The outer diameters a and c may be increased or decreased to adjust the overall flexibility of the combined shaft 48 and inner shaft 58 at the proximal curve 65 and that of the inner shaft 58 at the fixation curve 66. Generally, the inner shaft 58 alone is more flexible at the fixation curve 66 than the combined inner shaft 58 and shaft 48 at the proximal curve 65. Furthermore, as the inner shaft 58 is provided with a smaller diameter than the shaft 48, the inner shaft 58 may be guided into vessels having a reduced diameter than would be possible with the shaft 48.

The inner shaft 58 may be more flexible, or have a lower durometer, than the shaft 48. For example, the inner shaft 58 may be from about 250 to about 72 durometer. At the proximal curve 65, the overall catheter assembly 10 assumes the rigidity of the shaft 48. However, the portion of the inner shaft 58 protruding beyond the tapering region 75 is less rigid and more easily maneuvered into the distal branch veins of the coronary sinus 56. Furthermore, tension exerted on the shaft 48 along the proximal curve 65 decreases the flexibility of the shaft 48 along the proximal curve 65. However, the flexibility of the inner shaft 58 at the fixation curve 66 remains generally the same. Thus, a deflected portion of the catheter assembly 10 is more rigid than the same portion when not deflected. The overall flexibility of the shaft 48 and inner shaft 58 at the proximal curve 65 when in an unbiased, undeflected configuration may be from about 25 to about 63 durometer. In the deflected position, in which the tensioning member 70 is tensioned, the overall rigidity of the proximal curve 65 may be from about 63 to about 72 durometer. The flexibility of the shaft 48 and inner shaft 58 may be varied along their lengths as well.

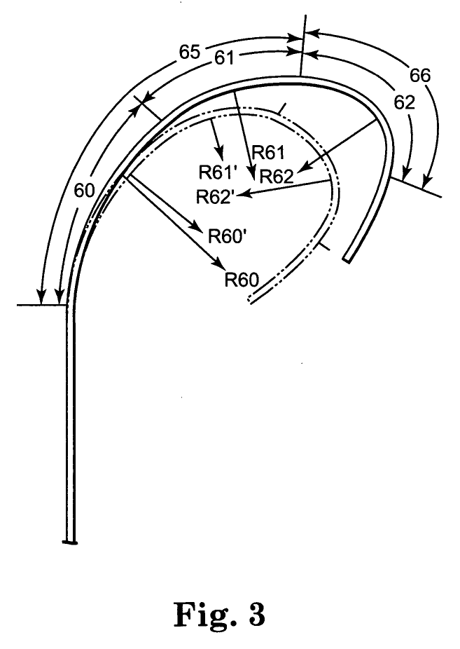

FIG. 3 shows a simplified view of the catheter assembly 10 of FIG. 2A in exemplary deflected and undeflected configurations. In the undeflected configuration, each segment 60, 61 and 62 has a radius of curvature R₆₀, R₆₁ and R₆₂, respectively. In the deflected configuration (shown in broken lines), the first and second segments 60, 61 are formed with sharper curves to have smaller radii of curvature R_{60′} and R_{61′}. The third segment radius of curvature R₆₂, however, is substantially the same in the deflected and undeflected configurations (i.e., R₆₂ is equal to R_{62′}).

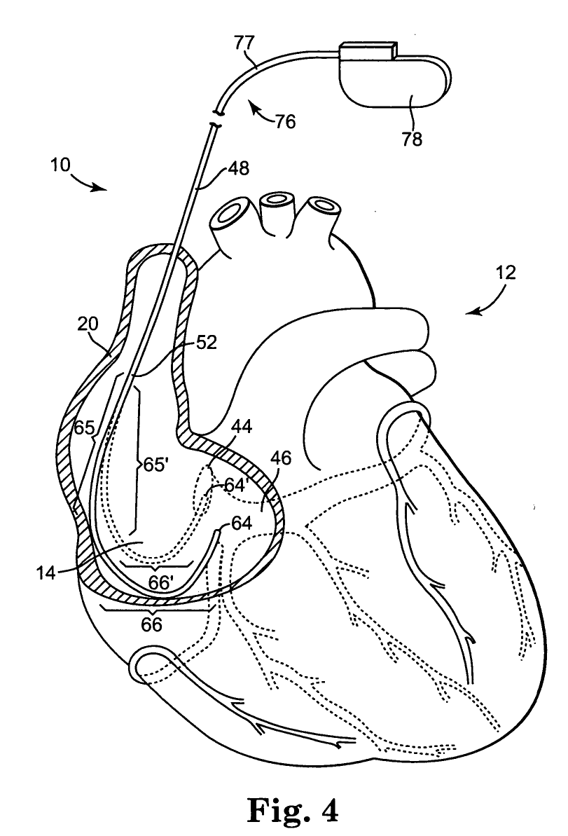

FIG. 4 shows a cardiac rhythm management (“CRM”) system 76 implanted in a heart 12 according to one embodiment of the present invention. The CRM system 76 includes a lead 77 coupled at a proximal end to a pulse generator 78 and coupled at a distal end to the heart 12. The lead 77 further includes an electrode at the distal end electronically coupled to the pulse generator 78 (not shown). As shown, the catheter assembly 10 is used to cannulate the coronary sinus 46 and serve as a guide for insertion of the distal end of the lead 77 into the coronary sinus 46. FIG. 4 shows the catheter assembly 10 of FIG. 2A in deflected and undeflected configurations while positioned in the heart 12. Generally, the curvature of the first and second segments 60 and 61 is such that the shaft 48 finds support during distal advancement from the walls of the right atrium 14. In the deflected configuration (shown in dashed lines and designated by prime numerals) the proximal curve 65 has been deflected to have a sharper curve. The distal tip 64 is thus selectively repositioned without significantly affecting the fixation curve 62. Further, the shaft 48 is no longer supported by the walls of the right atrium 14. The increased stiffness of the shaft 48 while the tensioning member 70 (not visible) is tensioned also provides more support as a payload, such the pacing lead 77, is advanced through the shaft 48 into the coronary sinus 46.

The catheter assembly 10 is distally advanced through the superior vena cava 20, the right atrium 14, the coronary sinus ostium 44, and into the coronary sinus 46. The catheter assembly 10 is further advanced so that the distal tip 64 of the shaft 48 is seated in a selected vessel, for example a side branch of the coronary sinus 46. Following insertion of the catheter assembly 10 into the coronary sinus 46, the lead 77 is inserted into the central lumen 57 and advanced distally within the inner shaft 58. Should the catheter tip 64 become dislodged, or the seated location prove inadequate, the tensioning member 70 may be tensioned to facilitate repositioning of the catheter assembly 10 without requiring removal of the lead 77 from the central lumen 57. Following insertion and seating of the lead 77 in a selected vessel, the catheter assembly 10 is removed and the lead 77 is coupled to the pulse generator 78, which is implanted under the skin in the chest.

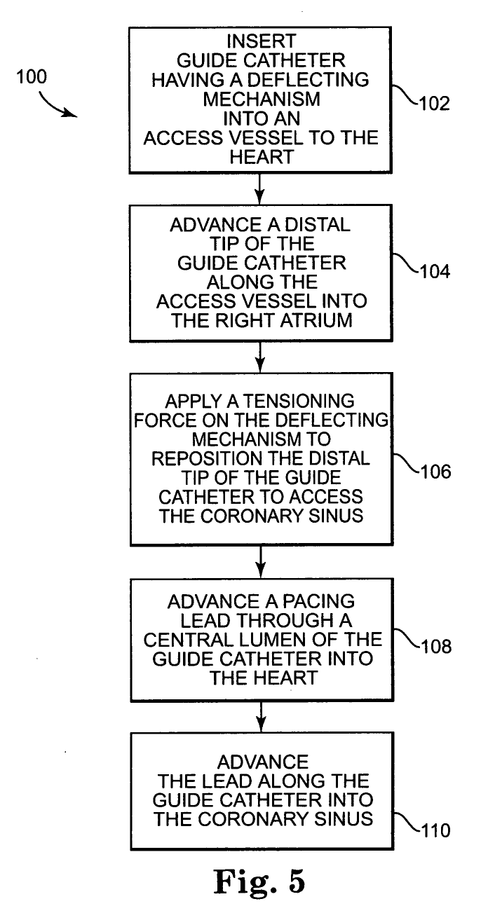

FIG. 5 is a flowchart summarizing a method 100 of cannulating the coronary sinus 46 according to one embodiment of the present invention. A guide catheter having a deflecting mechanism is inserted into an access vessel to the heart (block 102). A distal tip of the guide catheter is advanced along the access vessel into the right atrium (block 104). Tension is exerted on the deflecting mechanism to reposition the distal tip of the guide catheter to access the coronary sinus (block 106). A pacing lead is inserted into a central lumen of the guide catheter and advanced along the guide catheter into the heart (block 108). The lead is advanced along the guide catheter into the coronary sinus (block 110). Alternately, the coronary sinus 56 is cannulated by the guide catheter after inserting the lead.

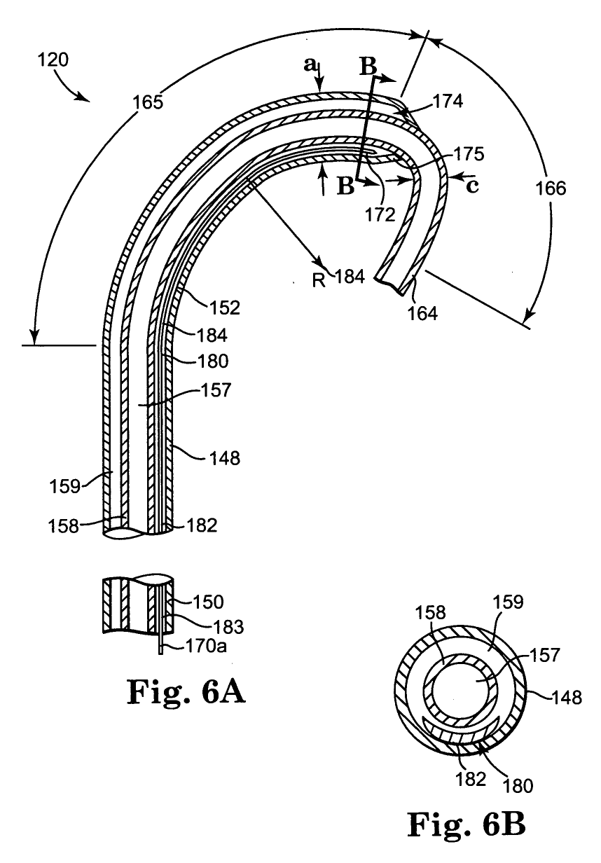

FIGS. 6A and 6B show a distal portion of a catheter assembly 120 according to another embodiment of the present invention. Catheter assembly 120 has many of the features of catheter assembly 10 as shown in FIG. 2A, and like parts are given like numbering. The catheter assembly 120 includes a flexible shaft 148 extending from a proximal end 150 to a distal portion 152, a central lumen 157 for receiving a pacing lead, and a secondary lumen 159. The distal portion 152 is pre-shaped with a proximal curve 165 and a distal fixation curve 166 extending distally therefrom. The secondary lumen 159 terminates at a shaft tapering region 175 proximal to the fixation curve 166. Catheter assembly 120 also includes a stylet deflection mechanism 180.

Stylet deflection mechanism 180 is a pre-shaped rigid member slidably receivable in the secondary lumen 159. The stylet deflection mechanism 180 includes a rigid, elongated stylet shaft 182 extending from a proximal end 183 to a distal portion 184. The distal portion 184 is curved and has a radius of curvature R₁₈₄. The stylet 180 is receivable into the secondary lumen 159 without removal of a payload, such as a pacing lead, from the central lumen 157.

FIG. 6B shows a cross-sectional view of the catheter assembly 120 of FIG. 6A taken along line B-B. The stylet shaft 182 has a generally crescent shaped cross sectional area. A crescent shaped cross-sectional area provides a more rigid shaft 182 having a greater bending moment than a similarly sized shaft having a circular cross sectional area. Therefore, a smaller crescent shaped shaft 182 may be employed which provides the same rigidity and bending moment as a larger circular shaft. The secondary lumen 159 need only accommodate the smaller crescent shaped shaft 182, and may be sized smaller than would be necessary to receive a circular stylet shaft sized to provide equal rigidity and bending moment. However, according to other embodiments of the present invention, the stylet shaft 182 may have a circular or other shaped cross-sectional area (not shown).

The stylet shaft 182 is sufficiently rigid to cause the catheter shaft 148 to conform to the shape of the stylet shaft 182. The termination of the secondary lumen 159 at the tapered region 175 functions as a stop, preventing over-insertion of the stylet deflection mechanism 180 into the fixation curve 166 of the catheter shaft 148.

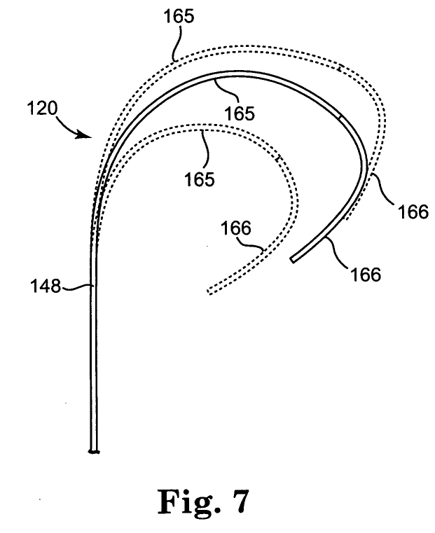

FIG. 7 shows a simplified view of the catheter assembly 120 of FIGS. 6A and 6B in various deflected positions. The catheter shaft 148 is shown in an undeflected position in the middle version. According to one embodiment, the radius of curvature R₁₈₄ of the distal portion 184 of the stylet shaft 182 (not visible) is greater than a radius of curvature of the proximal curve 165. When inserted into the catheter shaft 148, as is shown in the uppermost version, the stylet shaft 182 deflects and partially straightens the proximal curve 165, increasing a radius of curvature of the proximal curve 165. According to another embodiment, the stylet radius of curvature R184 is smaller than a radius of curvature of the proximal curve 165. Upon insertion into the catheter shaft 148, as is shown in the lowermost version, the stylet shaft 182 deflects the proximal curve 165, reducing a radius of curvature of the proximal curve 165.

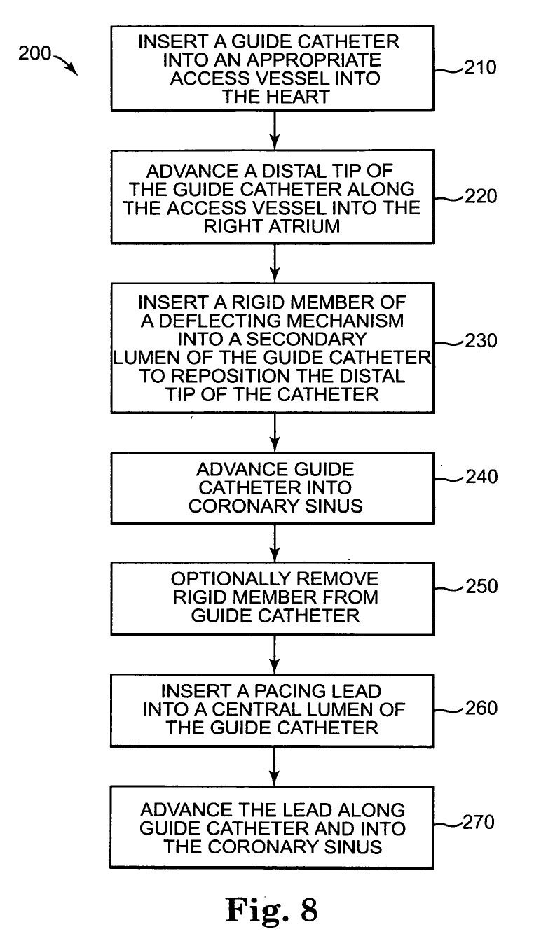

FIG. 8 summarizes a method 200 of cannulating the coronary sinus 46 according to one embodiment of the present invention. A guide catheter is inserted into an appropriate access vessel into the heart (block 210). A distal tip of the guide catheter is advanced along the access vessel into the right atrium (block 220). A rigid member of a deflecting mechanism is inserted into a secondary lumen of the guide catheter to reposition the distal tip of the catheter to access the coronary sinus (block 230). The guide catheter is advanced into the coronary sinus (block 240). The rigid member is optionally removed from the guide catheter (block 250). A pacing lead is inserted into a central lumen of the guide catheter (block 260). The lead is advanced along the guide catheter into the coronary sinus (block 270). Alternately, the rigid member is inserted into the guide catheter to reposition the distal tip of the catheter after the lead has been inserted into the guide catheter.

Sometimes, due to unusual patient physiology or disease, it is necessary to adjust the curvature of the distal portion 52 of the shaft 148 to access the coronary sinus 46. Sometimes, it may not be desirable to wrap around or find support from the walls of the right atrium 14 because of patient physiology or disease. Sometimes, it is desirable to reposition the distal tip 164 of the shaft 148 to make effective use of the fixation curve 166 to access the coronary sinus 46.

A catheter assembly 120 according to the present embodiment permits adjustability of the curvature and flexibility of the shaft 148 at the proximal curve 165 without significantly affecting the flexibility and curvature of the shaft 148 at the distal curve 166. Reduced flexibility of the shaft 148 at the proximal curve 165 increases the support and maneuverability of the catheter assembly 120. The distal curve 166, however, remains flexible as the catheter assembly 120 cannulates the heart 12, and retains a pre-formed curvature chosen to facilitate access to a destination vessel, for example, the coronary sinus 46. Furthermore, because the stylet deflecting mechanism 180 is positioned in secondary lumen 159, which is separate from the central lumen 157, the stylet deflecting mechanism 180 is operable whether or not a payload such as a lead has already been inserted into the central lumen 157. Thus, if it becomes necessary to reposition the shaft 148 after the payload has been inserted into the central lumen 157, the stylet deflecting mechanism 180 may be operated without removing the payload. The stylet deflecting mechanism 180 may also be operated to retain the shaft 148 in a particular configuration or to provide support as a payload is advanced through the central lumen 157.

Both the tensioning member 70 described with respect to the embodiment shown generally in FIG. 2A and the rigid member 182 described with respect to the embodiment shown generally in FIG. 6A are deflecting means for deflecting the shaft and more particularly for deflecting a first proximal segment of the shaft from a first configuration to a second configuration without altering the curvature of a second distal segment.

According to another embodiment, a catheter assembly is provided with both a tensioning deflecting mechanism as shown in FIG. 2A and a stylet deflecting mechanism as shown in 6A (not shown).

Various modifications and additions can be made to the exemplary embodiments discussed without departing from the scope of the present invention. Accordingly, the scope of the present invention is intended to embrace all such alternatives, modifications, and variations as fall within the scope of the claims, together with all equivalents thereof.

I claim:

1. A catheter assembly for cannulating a coronary sinus of the heart, the catheter assembly comprising:
a flexible, elongated shaft having:
a proximal end,
a distal end preformed with a first curved segment and a second curved segment extending distal to the first curved segment,
a central lumen, and
a secondary lumen co-axial with the central lumen; and
a tensioning member positioned in the secondary lumen and coupled to the shaft proximal to the second curved segment for deflecting the first curved segment from a first configuration to a second configuration.

2. The catheter assembly of claim 1 wherein the first curved segment has a first radius of curvature in the first position and a second radius of curvature in the second position.

3. The catheter assembly of claim 1 wherein the shaft at the first curved segment is more rigid in the second configuration than in the first configuration.

4. The catheter assembly of claim 1 wherein the shaft has a first outer diameter at the first curved segment and tapers at a tapering region to a second outer diameter at the second curved segment, the second outer diameter having a smaller magnitude that the first outer diameter.

5. The catheter assembly of claim 1 wherein the shaft further comprises an inner shaft and an outer shaft, wherein the inner shaft is positioned with a lumen of the outer shaft, and wherein the secondary lumen is an annular space defined between the inner shaft and the outer shaft.

6. The catheter assembly of claim 5 wherein the outer shaft terminates at the tapering region.

7. The catheter assembly of claim 1, wherein the deflection mechanism further comprises a rigid member having a curved distal end sized to be slidably receivable in the secondary lumen.

8. The catheter assembly of claim 7 wherein the distal end of the rigid member has a radius of curvature greater than a radius of curvature of the first curved segment in the first configuration.

9. The catheter assembly of claim 7 wherein the distal end of the rigid member has a radius of curvature smaller than a radius of curvature of the first curved segment in the first configuration.

10. The catheter assembly of claim 7 wherein the rigid member has a crescent shaped cross sectional area.

11. The catheter assembly of claim 7 wherein the rigid member has a circular cross sectional area.

12. A system for performing cardiac rhythm management on a heart, the system comprising:
a pulse generator;
a lead having a proximal end coupled to the pulse generator, a distal end coupled to the heart and an electrode electrically coupled to the pulse generator at the distal end of the lead; and
a catheter assembly adapted for positioning the distal end of the lead in a coronary vein of the heart, the catheter assembly comprising:
a flexible, elongated shaft having:
a proximal end,
a distal end preformed with a first curved segment and a second curved segment extending distal to the first curved segment,
a central lumen, and
a secondary lumen co-axial with the central lumen; and
a deflecting means for deflecting the first curved segment from a first configuration to a second configuration.

13. The catheter assembly of claim 12 wherein the deflecting means further comprises a tensioning member extending through the secondary lumen, the tensioning member coupled at a distal end to the shaft proximal to the second curved segment.

14. The catheter assembly of claim 12 wherein the deflecting means further comprises a rigid member sized to be slidably receivable in the secondary lumen, the rigid member having a curved distal end.

15. A method of cannulating the coronary sinus, the method comprising:
providing a catheter assembly of the type including a shaft having a proximal end, a distal end provided with a first curved segment and a second curved segment extending distally therefrom, a central lumen for receiving a pacing lead and a deflecting mechanism positioned in a secondary lumen of the shaft;
inserting the distal end of the shaft into an access vessel to the heart;
advancing the catheter assembly distally along the access vessel;
deflecting the first curved segment to reposition the shaft while allowing the second curved segment to remain in substantially the same configuration;
accessing the coronary sinus with the shaft; and
advancing a pacing lead into the coronary sinus through the central lumen.

16. The method of claim 15 wherein deflecting the first curved segment further includes reducing a radius of curvature of the first curved segment.

17. The method of claim 15 wherein deflecting the first curved segment further includes increasing a radius of curvature of the first curved segment.

18. The method of claim 15 wherein deflecting the first curved segment further includes tensioning a tensioning member coupled at a distal end to the guide catheter proximal to the second curved segment.

19. The method of claim 15 wherein deflecting the first curved segment further includes inserting a rigid member into the guide catheter.

20. The method of claim 15 wherein the pacing lead is advanced through the guide catheter prior to deflecting the first curved segment.
